(19) United States
(12) Patent Application Publication
(10) Pub. No.: US 2010/0041653 A1
(43) Pub. Date: Feb. 18, 2010

(54) COMPOSITION AND METHODS FOR THE DESIGN AND DEVELOPMENT OF METALLO-ENZYME INHIBITORS

(75) Inventors: Maurizio Pellecchia, San Diego, CA (US); Seth M. Cohen, San Marcos, CA (US)

Correspondence Address: DLA PIPER LLP (US), 4365 EXECUTIVE DRIVE, SUITE 1100, SAN DIEGO, CA, 92121-2133, US

(73) Assignees: BURNHAM INSTITUTE FOR MEDICAL RESEARCH, La Jolla, CA (US); THE REGENTS OF THE UNIVERSITY OF CALIFORNIA, Oakland, CA (US)

(21) Appl. No.: 12/541,871

(22) Filed: Aug. 14, 2009

Related U.S. Application Data
(60) Provisional application No. 61/089,364, filed on Aug. 15, 2008.

(51) Int. Cl. A61K 31/5377; A61K 31/4965; A61K 31/505; A61K 31/44; C07D 239/02; C07D 241/02; C07D 213/78; C07D 413/04; A61K 31/47; C07D 215/00

(52) U.S. Cl. 514/235.5; 514/255.06; 514/256; 514/350; 544/316; 544/406; 546/298; 544/131; 514/311; 546/152

(57) ABSTRACT

The present disclosure provides compounds having the general structure A or pharmaceutically acceptable salts thereof:

R—X (A)

wherein R is an alkyl or aryl moiety comprising heterocyclic structures; and X is a metal-chelatin group selected from:

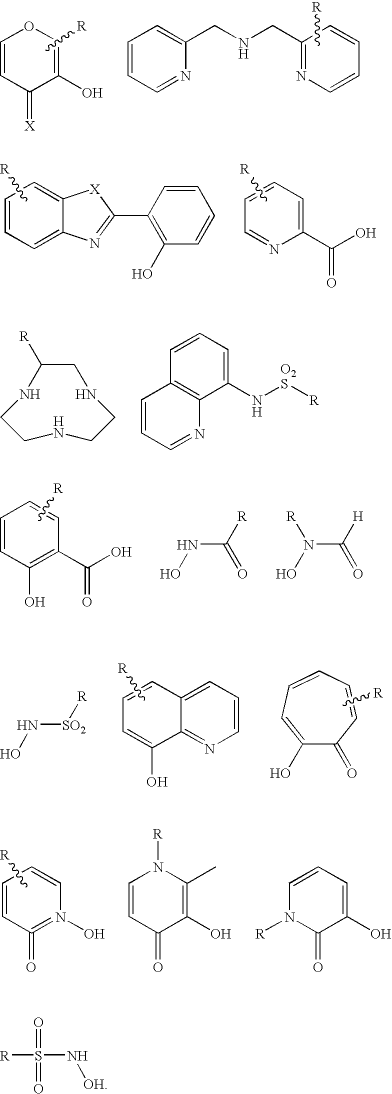

This disclosure further provides a focused library of compounds for use in the discovery and design of metallo-enzyme inhibitors. This fragment-based approach provides an assembly of a library of low molecular weight compounds (MW<300 Da) containing a variety of potential metal-chelating groups. The identification of the inhibitory scaffolds among these compounds provides the initial hit fragments that may be optimized for affinity against a particular target using common medicinal chemistry, structure-based or NMR-based approaches.

### CROSS REFERENCE TO RELATED APPLICATION

This application claims the benefit of priority under 35 U.S.C. §119(e) of U.S. Ser. No. 61/089,364, filed Aug. 15, 2008, the entire content of which is incorporated herein by reference.

### FIELD OF THE DISCLOSURE

This disclosure describes the composition of a library of compounds and their use for the discovery and design of metallo-enzyme inhibitors.

### BACKGROUND

The discovery of inhibitors of metallo-enzymes, such as metallo-proteases for example, has not been particularly successful by direct high-throughput (HTS) screening approaches. This is due to the highly specific nature of the active site of the enzymes that generally require a metal-chelating group for an inhibitor to be efficient.

### SUMMARY

Here we report on a fragment-based approach that consists first on the design, synthesis and assembly of a library of low molecular weight compounds (MW<300 Da) all containing a variety of potential metal-chelating groups. The identification of preferential inhibitory scaffolds among these compounds will lead to initial hit fragments that are optimized for affinity against a particular target using common medicinal chemistry, structure-based or NMR-based approaches. We report on the design, synthesis and applications of a focused fragment library in which each compound contains a diversity element and a metal-chelating moiety.

According to embodiments of the present disclosure, there are provided compounds having the general structure A or pharmaceutically acceptable salts thereof:

R—X (A)

wherein R is an alkyl or aryl moiety comprising heterocyclic structures; and X is a metal chelating structure among one of these listed below:

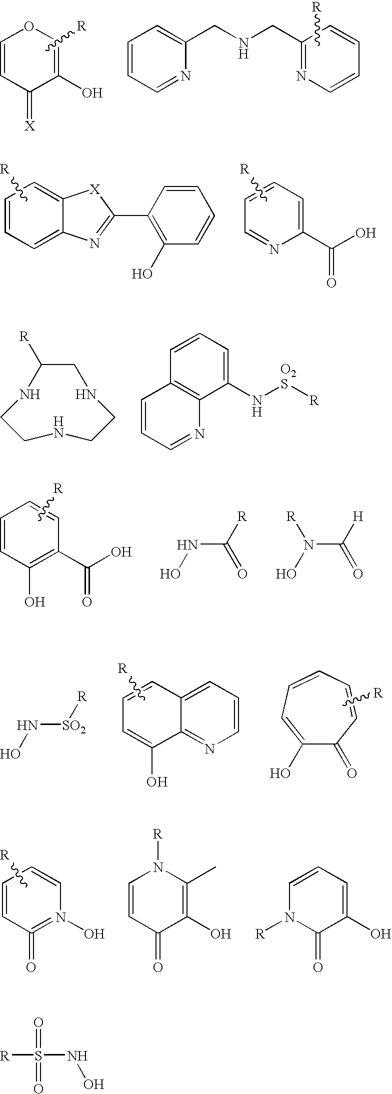

According to some embodiments of the present disclosure, compounds are provided having the formulae reported below:

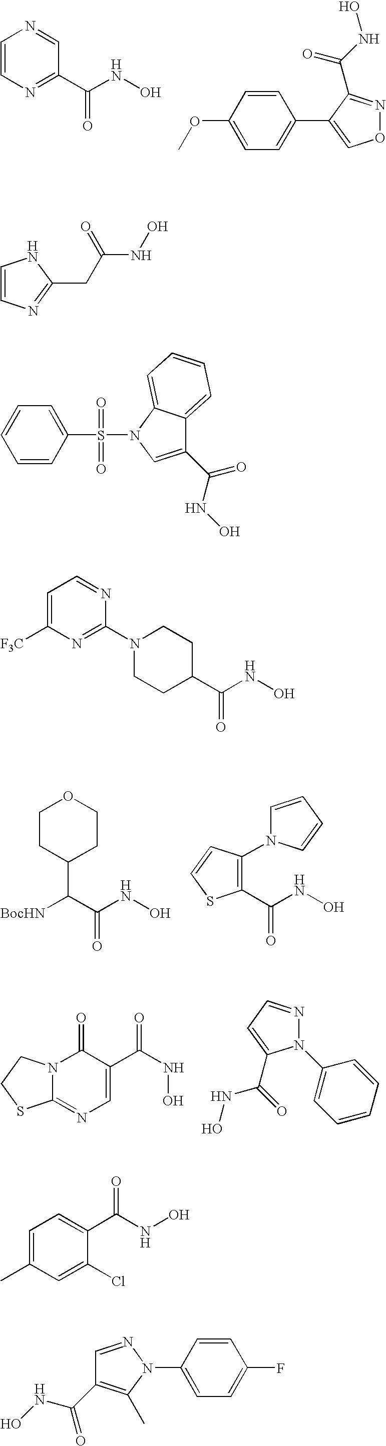

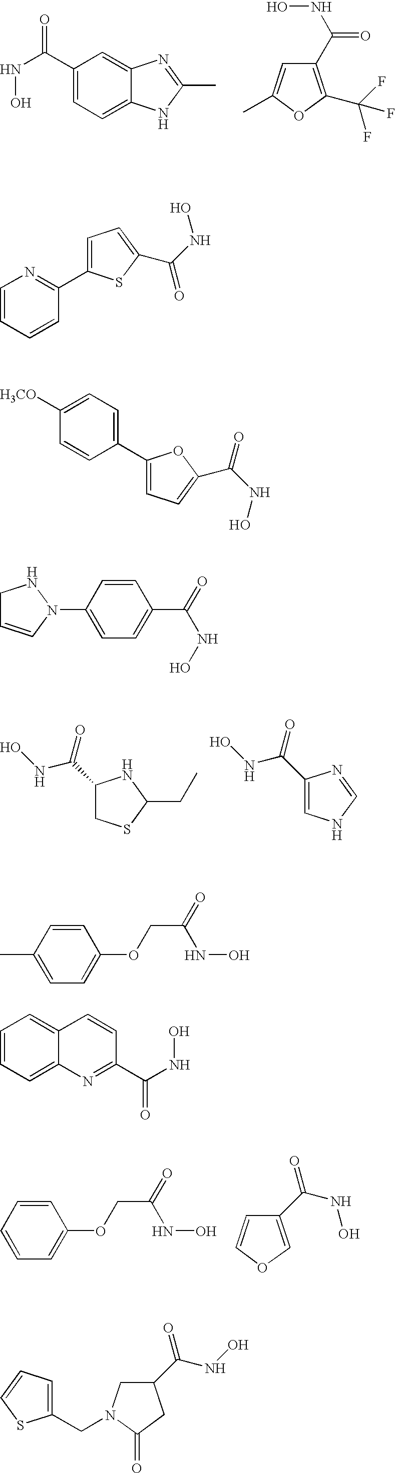

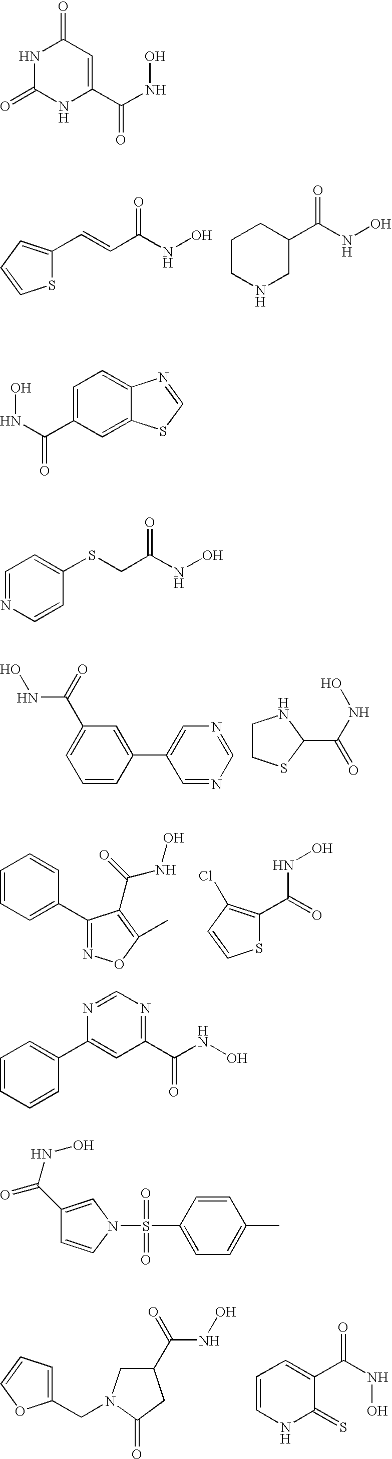

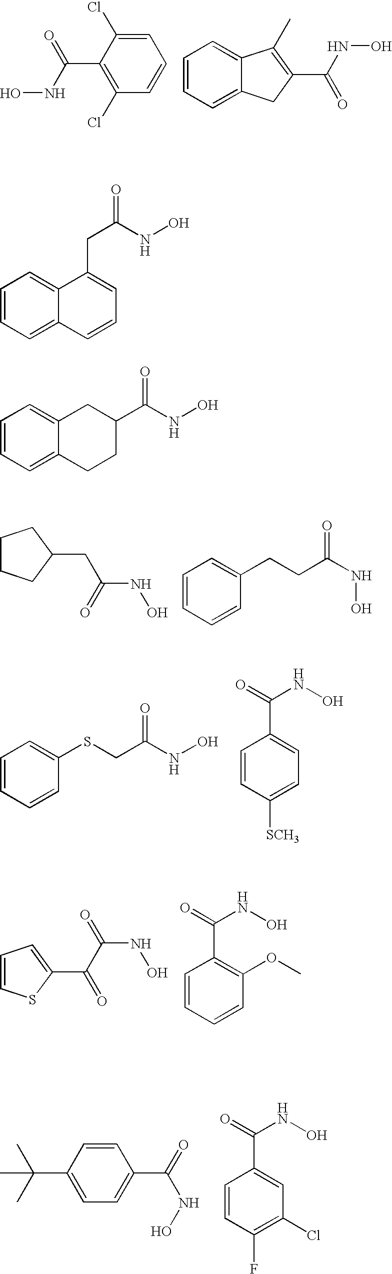

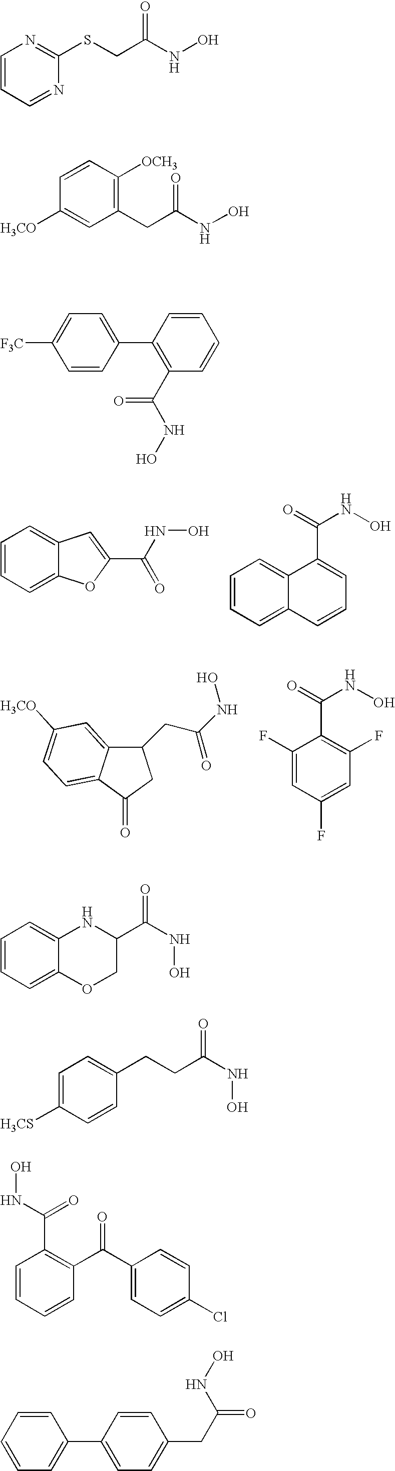

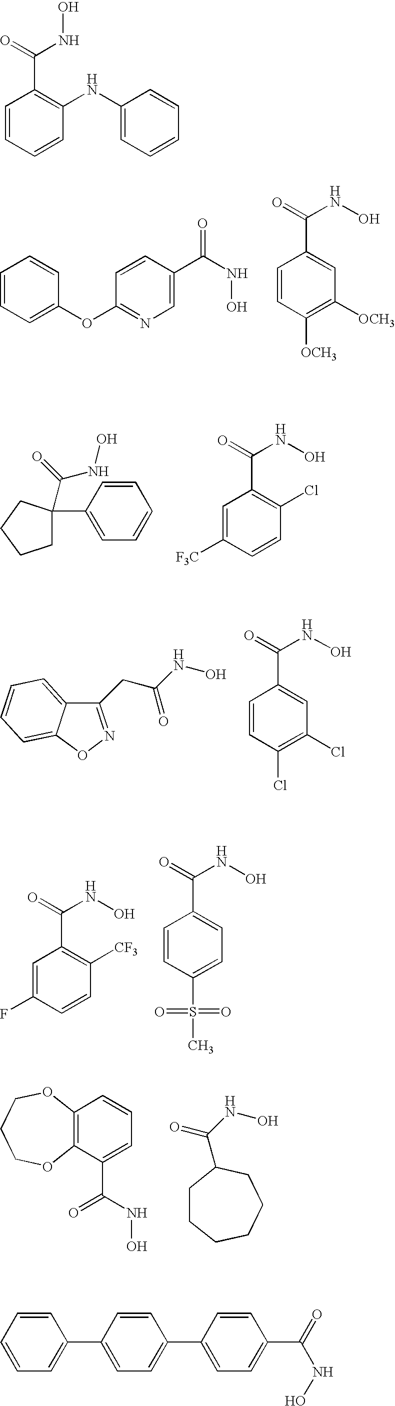

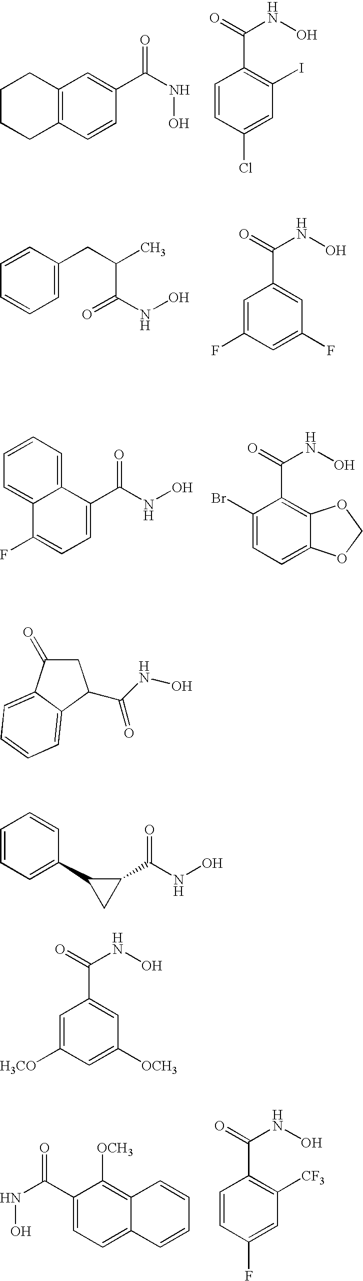

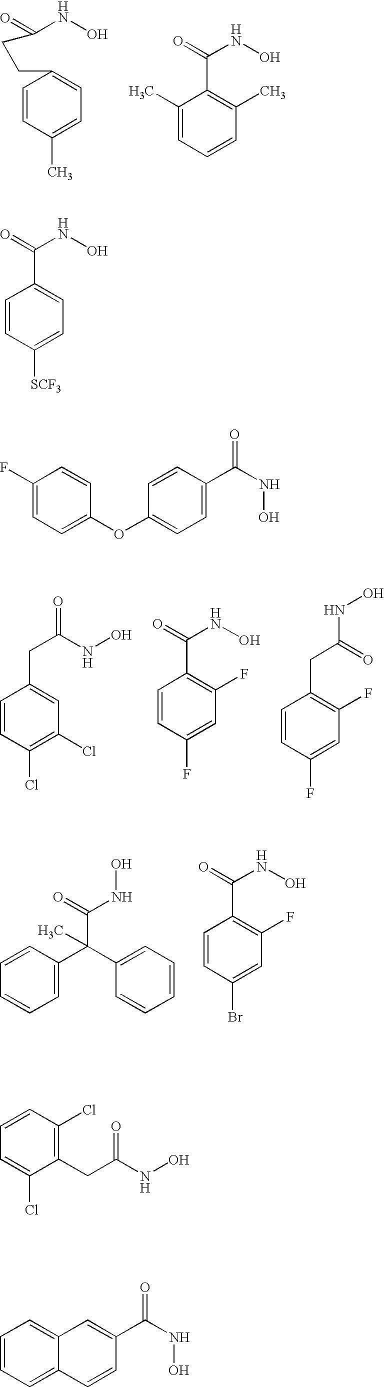

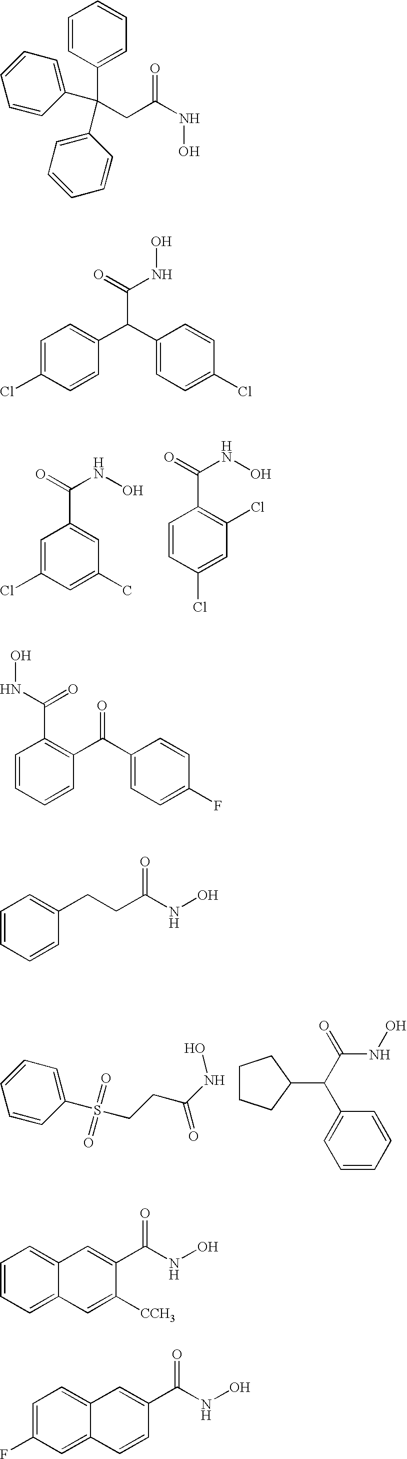

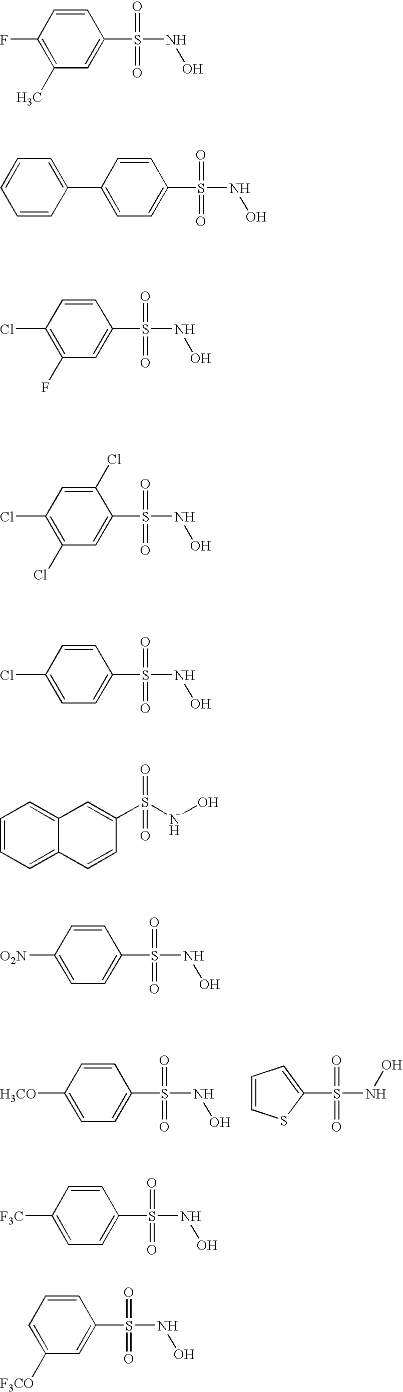

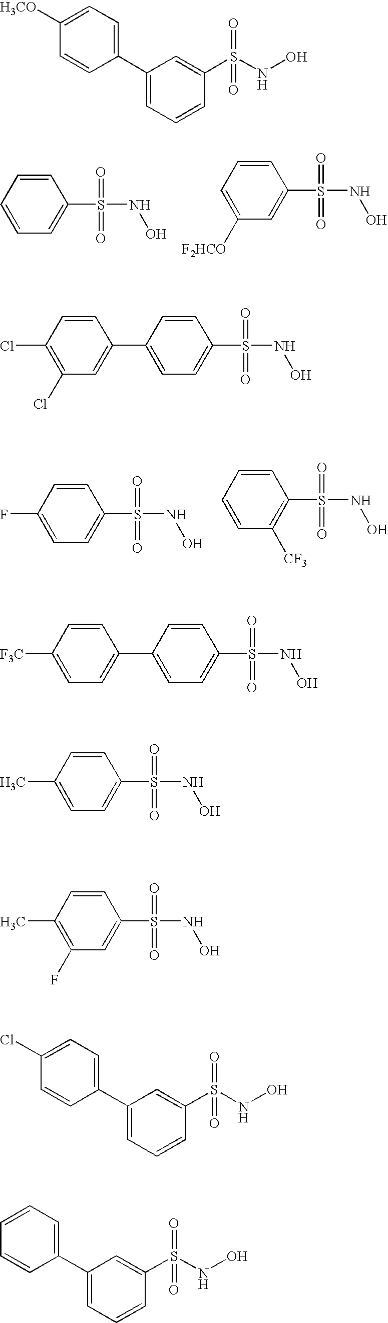

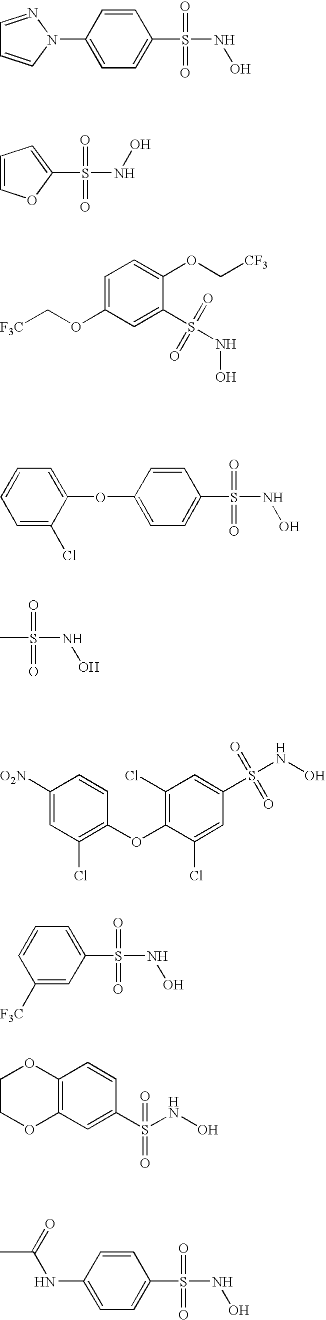

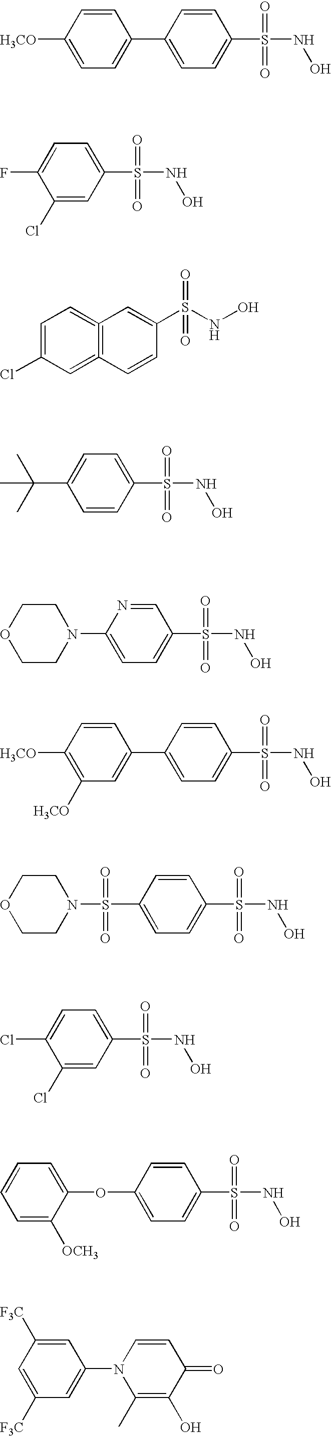

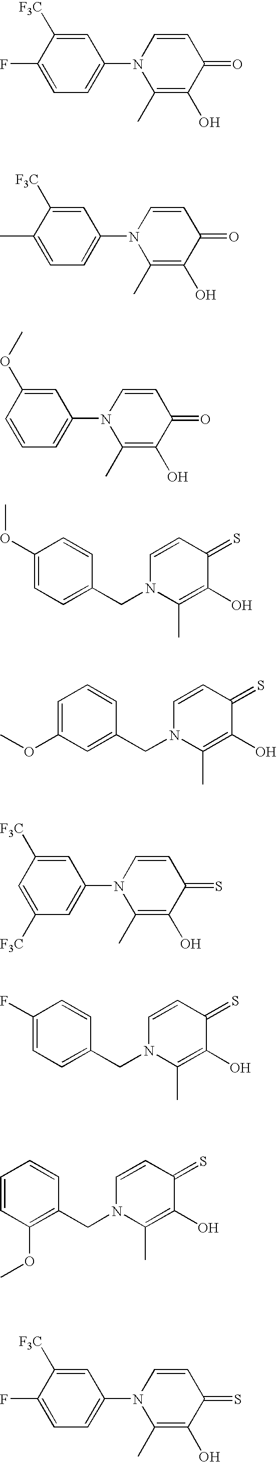

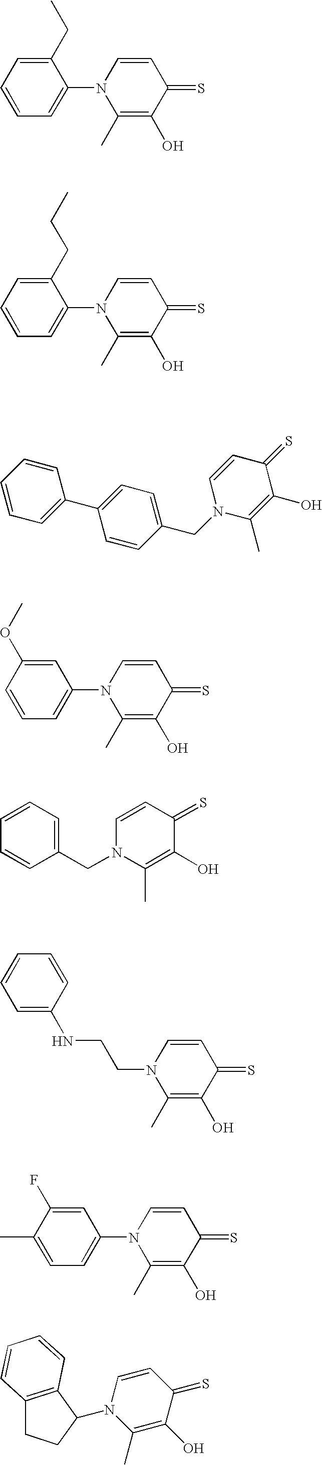

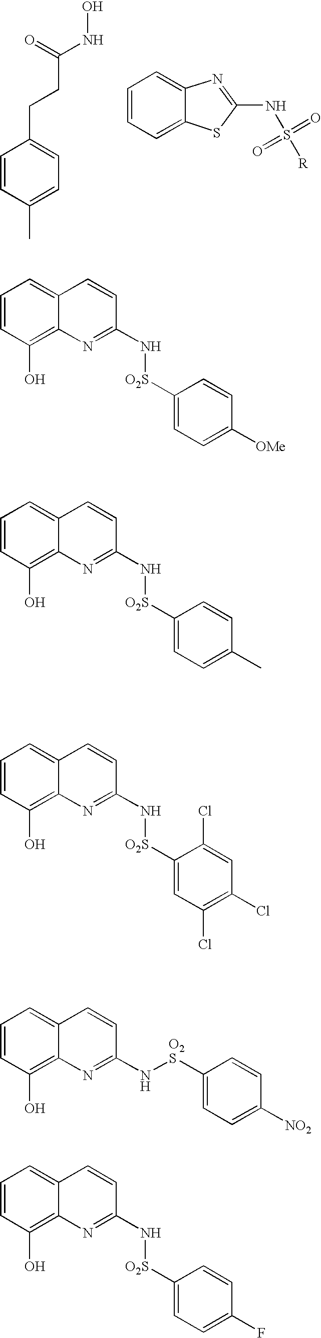

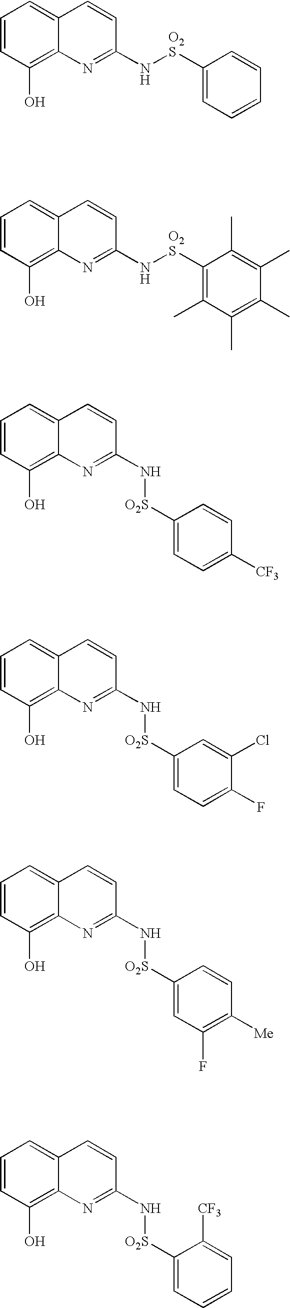

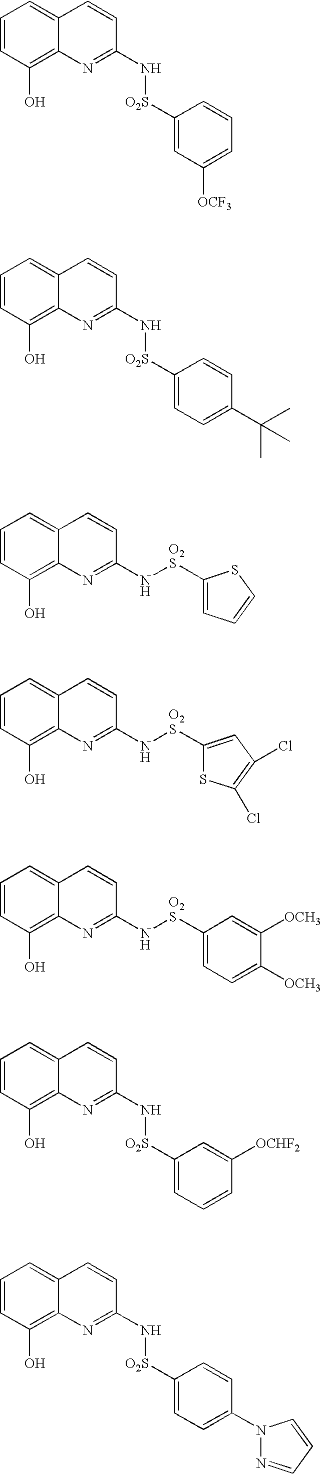

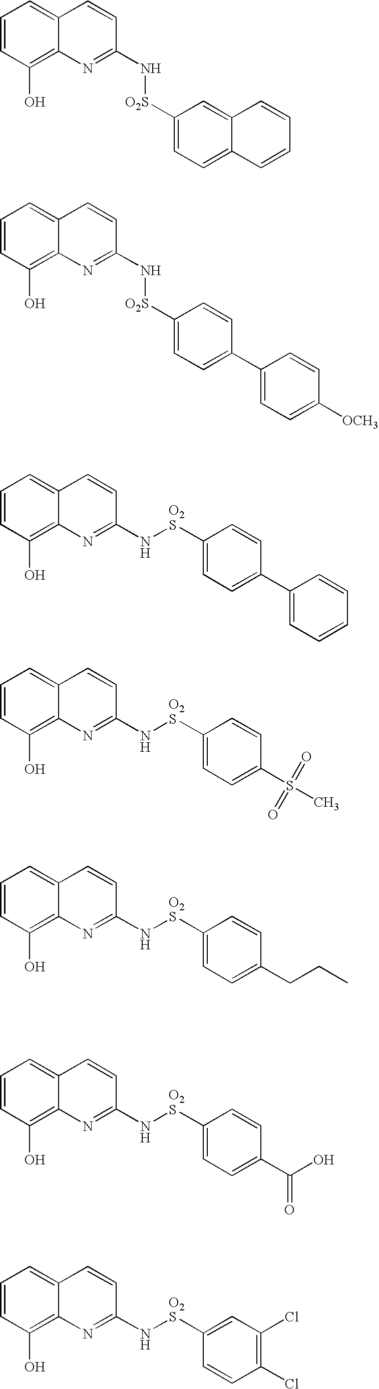

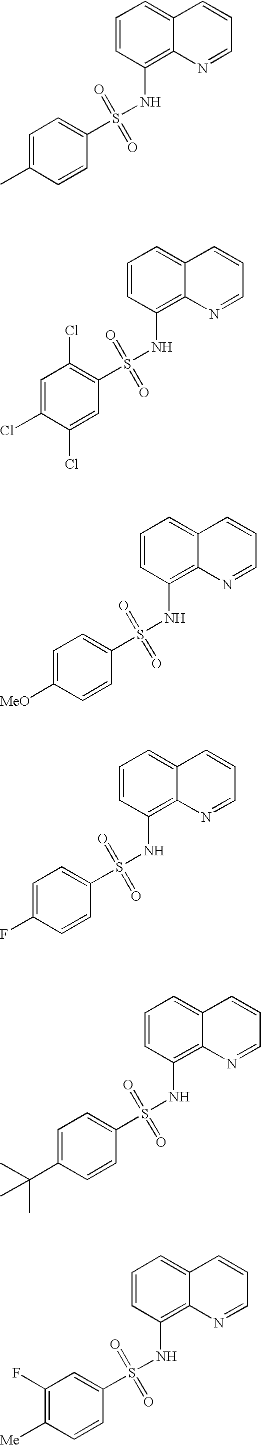

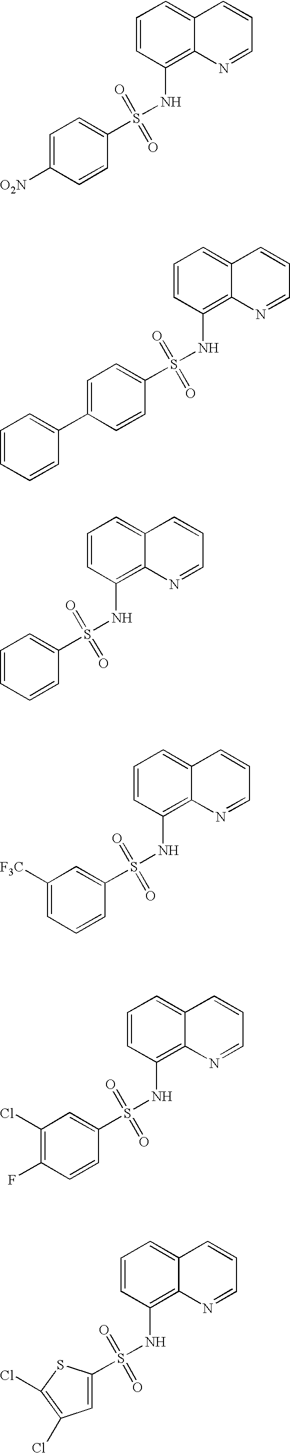

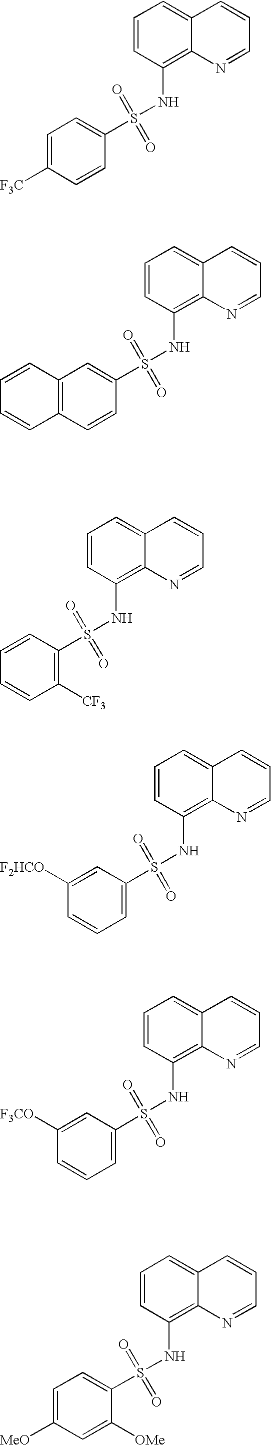

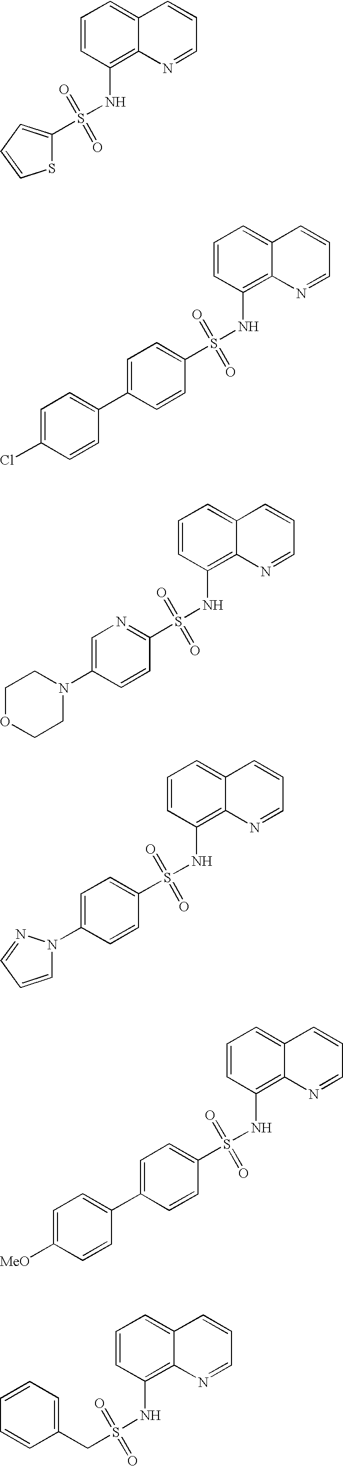

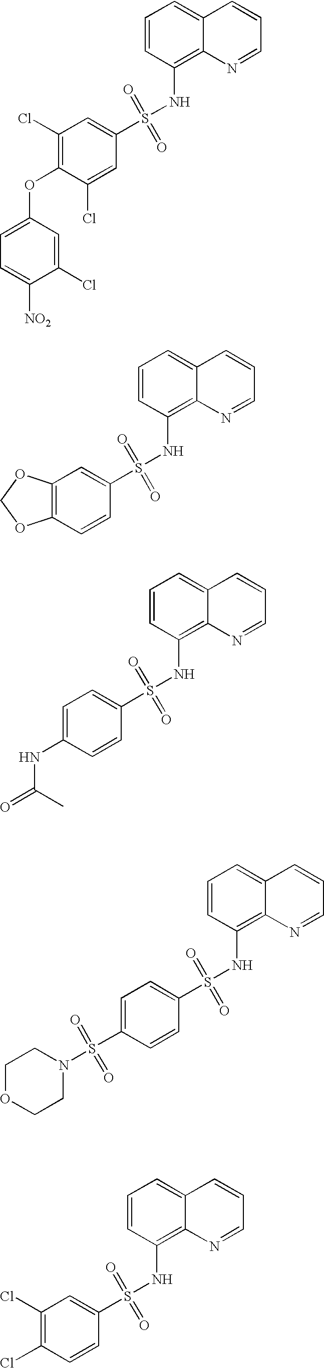

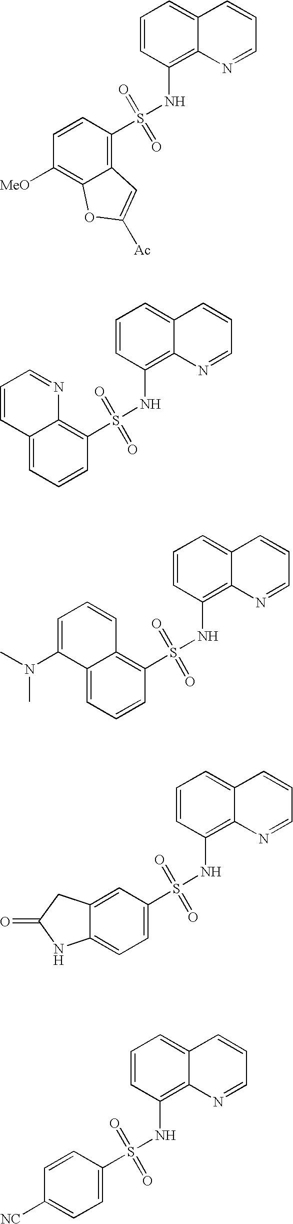

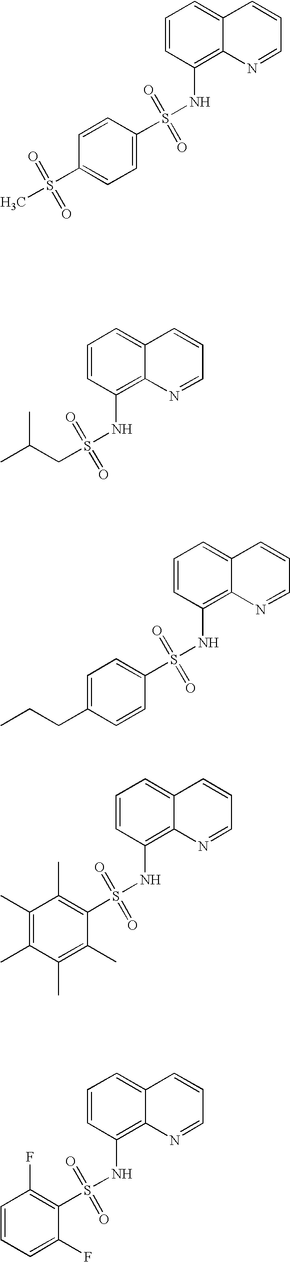

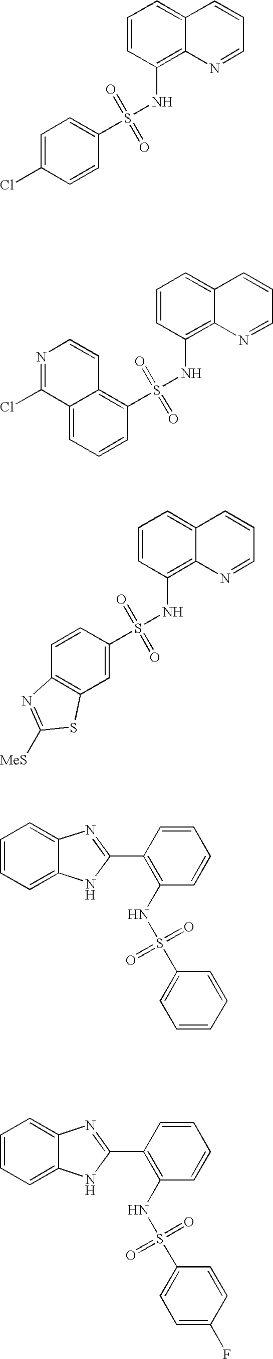

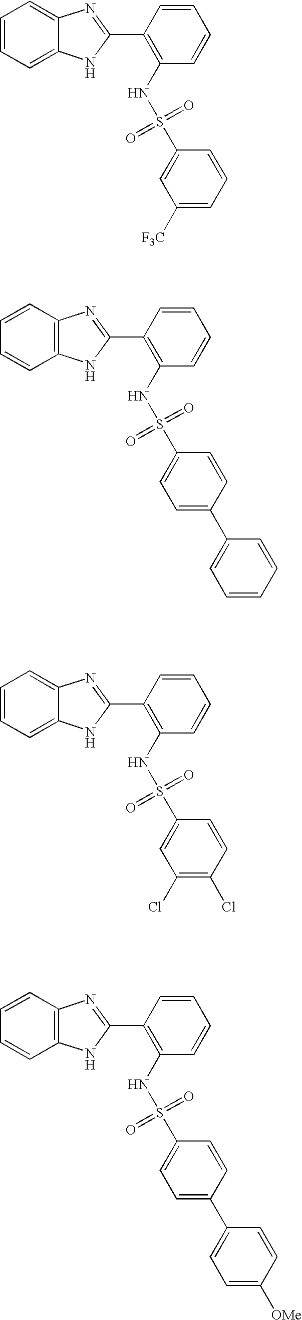

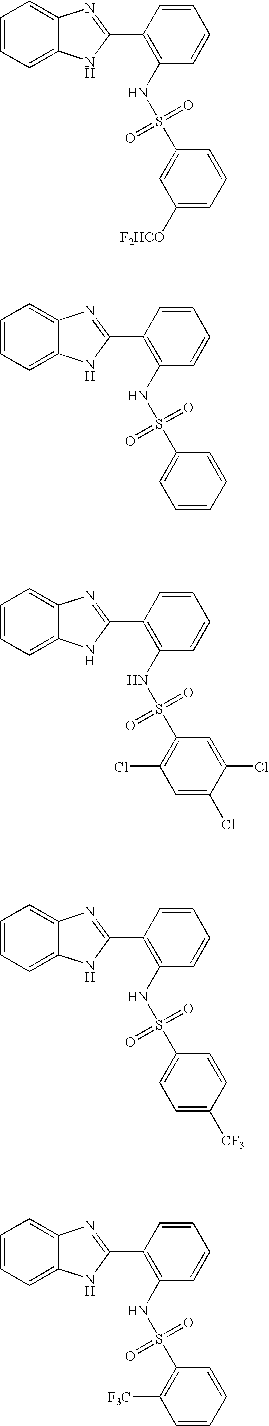

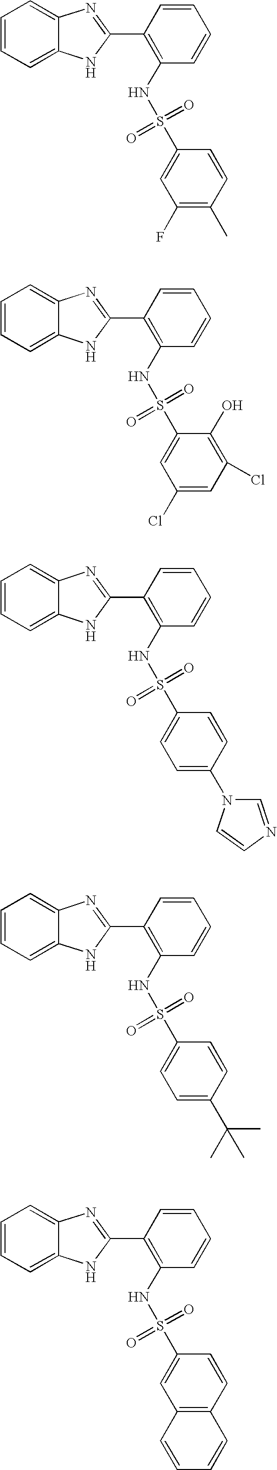

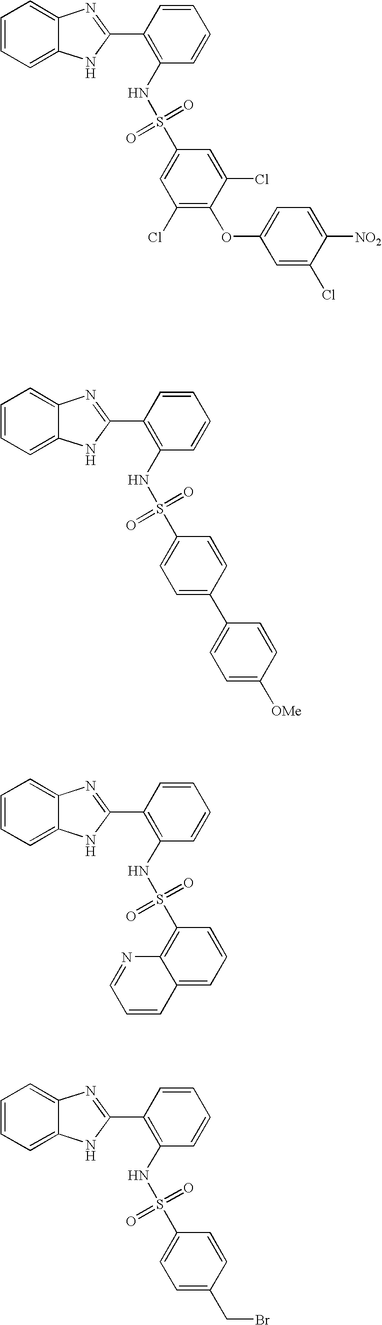

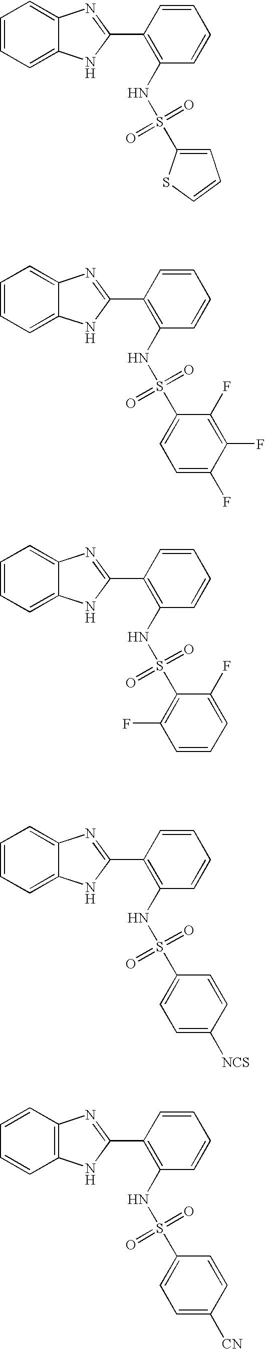

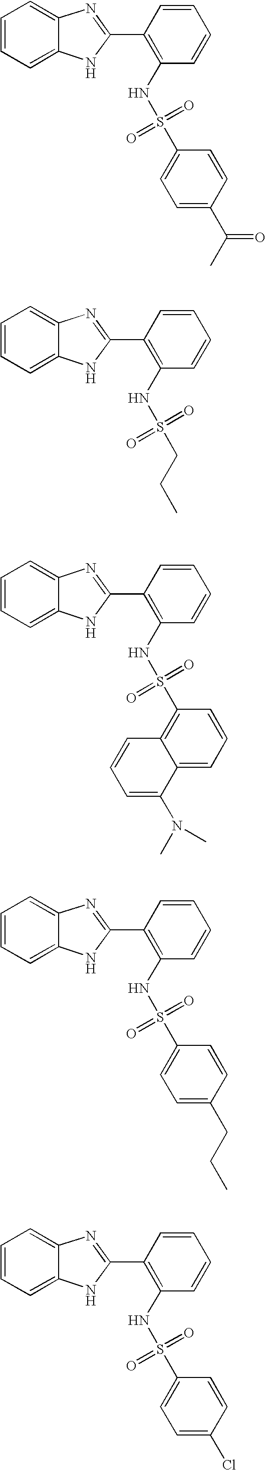

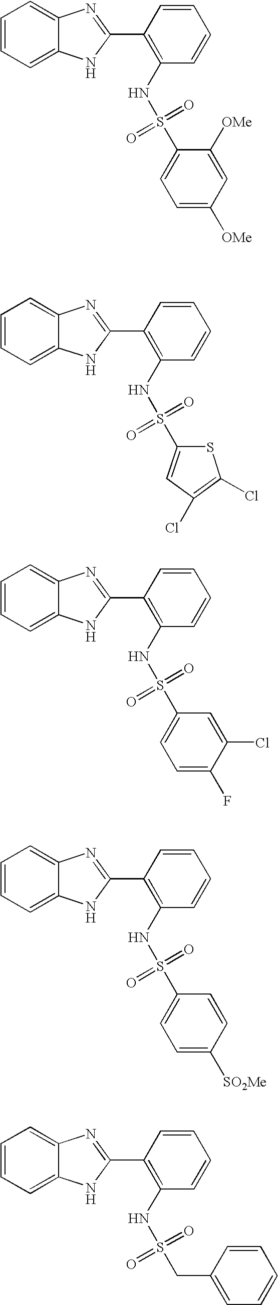

According to other embodiments of the present disclosure, initial library elements with general structure A can be identified as inhibitors of a given metallo-enzyme.

According to other embodiments of the present disclosure, methods for the treatment of human malignancies, methods comprising administering to a subject in need thereof a pharmacologically effective dose of a pharmaceutical composition comprising a compound having the general structure A.

### BRIEF DESCRIPTION OF THE EXAMPLES

EXAMPLE 1 illustrates the scheme used for synthesis of some of the compounds listed.

EXAMPLE 2 illustrates the scheme used for synthesis of some of the compounds listed.

EXAMPLE 3 illustrates the scheme used for synthesis of some of the compounds listed.

### DETAILED DESCRIPTION

The following definitions are used, unless otherwise described.

The term “alkyl” refers to either substituted or unsubstituted C₁-C₁₀ straight chain saturated aliphatic hydrocarbon groups, substituted and unsubstituted C₂-C₁₀ straight chain unsaturated aliphatic hydrocarbon groups, substituted and unsubstituted C₄-C₁₀ branched saturated aliphatic hydrocarbon groups, substituted and unsubstituted C₄-C₁₀ branched unsaturated aliphatic hydrocarbon groups, substituted and unsubstituted C₃-C₈ cyclic saturated aliphatic hydrocarbon groups, substituted and unsubstituted C₅-C₈ cyclic unsaturated aliphatic hydrocarbon groups having the specified number of carbon atoms. For example, the definition of “alkyl” shall include but is not limited to: methyl, ethyl, propyl, butyl, pentyl, hexyl, heptyl, octyl, nonyl, decyl, undecyl, ethenyl, propenyl, butenyl, penentyl, hexenyl, heptenyl, octenyl, nonenyl, decenyl, undecenyl, isopropyl, isobutyl, tert-butyl, sec-butyl, isopentyl, neopentyl, cyclopropyl, cyclobutyl, cyclopentyl, cyclohexyl, cycloheptyl, cyclooctyl, cyclopentenyl, cyclohexenyl, cycloheptenyl, cyclooctenyl, methylcyclopropyl, ethylcyclohexenyl, butenylcyclopentyl, adamantyl, norbornyl and the like.

Alkyl substituents are independently selected from a group consisting of halogen, —OH, —SH, —NH₂, —CN, —NO₂, ═O, ═CH₂, trihalomethyl, carbamoyl, arylC₀₋₁₀alkyl, heteroarylC₀₋₁₀alkyl, C₁₋₁₀alkyloxy, arylC₀₋₁₀alkyloxy, C₁₋₁₀alkylthio, arylC₀₋₁₀alkylthio, C₁₋₁₀alkylamino, arylC₀₋₁₀alkylamino, N-aryl-N—C₀₋₁₀alkylamino, C₁₋₁₀alkylcarbonyl, arylC₀₋₁₀alkylcarbonyl, C₁₋₁₀alkylcarboxy, arylC₀₋₁₀alkylcarboxy, C₁₋₁₀alkylcarbonylamino, arylC₀₋₁₀alkylcarbonylamino, tetrahydrofuryl, morpholinyl, piperazinyl, hydroxypyronyl, —C₀₋₁₀alkylCOORₐ and —C₀₋₁₀alkylCONR_{b}R_{c}, wherein Rₐ, R_{b} and R_{c} are independently selected from hydrogen, alkyl, aryl, or R_{b} and R_{c} are taken together with the nitrogen to which they are attached forming a saturated cyclic or unsaturated cyclic system containing 3 to 8 carbon atoms, with at least one substituent.

The term “aryl” refers to an unsubstituted, mono-, di- or trisubstituted monocyclic, polycyclic, biaryl aromatic groups covalently attached at any ring position capable of forming a stable covalent bond, certain points of attachment being apparent to those skilled in the art (e.g., 3-phenyl, 4-naphtyl and the like). The aryl substituents are independently selected from a group consisting of halogen, —OH, —SH, —CN, —NO₂, trihalomethyl, hydroxypyronyl, C₁₋₁₀alkyl, arylC₀₋₁₀alkyl, C₀₋₁₀alkyloxyC₀₋₁₀alkyl, arylC₀₋₁₀alkyloxyC₀₋₁₀alkyl, C₀₋₁₀alkylthio-C₀₋₁₀alkyl, arylC₀₋₁₀alkylthioC₀₋₁₀alkyl, C₀₋₁₀alkylaminoC₀₋₁₀alkyl, arylC₀₋₁₀alkylamino-C₀₋₁₀alkyl, N-aryl-N—C₀₋₁₀alkylaminoC₀₋₁₀alkyl, C₁₋₁₀alkylcarbonylC₀₋₁₀alkyl, arylC₀₋₁₀alkyl-carbonylC₀₋₁₀alkyl, C₁₋₁₀alkylcarboxyC₀₋₁₀alkyl, arylC₀₋₁₀alkylcarboxyC₀₋₁₀alkyl, C₁₋₁₀alkyl-carbonyl-aminoC₀₋₁₀alkyl, arylC₀₋₁₀alkylcarbonylaminoC₀₋₁₀alkyl, —C₀₋₁₀alkylCOORₐ, and —C₀₋₁₀alkyl-CONR_{b}R_{c}, wherein Rₐ, R_{b} and R_{c} are independently selected from hydrogen, alkyl, aryl or R_{b} and R_{c} are taken together with the nitrogen to which they are attached forming a saturated cyclic or unsaturated cyclic system containing 3 to 8 carbon atoms with at least one substituent.

The definition of “aryl” includes, but is not limited to, such specific groups as phenyl, biphenyl, naphthyl, dihydronaphthyl, tetrahydronaphthyl, indenyl, indanyl, azulenyl, anthryl, phenanthryl, fluorenyl, pyrenyl and the like.

The terms “heteroaryl”, “heterocycle” or “heterocyclic” refer to a monovalent unsaturated group having a single ring or multiple condensed (also known as “fused”) rings, from 1 to 8 carbon atoms and from 1 to 4 hetero atoms selected from nitrogen, sulfur or oxygen within the ring. The heteroaryl groups in this disclosure can be optionally substituted with 1 to 3 substituents selected from a group consisting of: halogen, —H, —SH, CN, —NO₂, trihalomethyl, hydroxypyronyl, C₁₋₁₀alkyl, arylC₀₋₁₀alkyl, C₀₋₁₀alkyloxyC₀₋₁₀alkyl, aryl-C₀₋₁₀alkyloxyC₀₋₁₀alkyl, C₀₋₁₀alkylthioC₀₋₁₀alkyl, arylC₀₋₁₀alkylthioC₀₋₁₀alkyl, C₀₋₁₀alkyl-aminoC₀₋₁₀alkyl, arylC₀₋₁₀alkylaminoC₀₋₁₀alkyl, N-aryl-N—C₀₋₁₀alkylaminoC₀₋₁₀alkyl, C₁₋₁₀alkylcarbonylC₀₋₁₀alkyl, arylC₀₋₁₀alkylcarbonylC₀₋₁₀alkyl, C₁₋₁₀alkylcarboxyC₀₋₁₀alkyl, arylC₀₋₁₀alkylcarboxyC₀₋₁₀alkyl, C₁₋₁₀alkylcarbonylaminoC₀₋₁₀alkyl, arylC₀₋₁₀alkylcarbonyl-aminoC₀₋₁₀alkyl, —C₀₋₁₀alkylCOORₐ, and —C₀₋₁₀alkylCONR_{b}R_{c}, wherein Rₐ, R_{b} and R_{c} are independently selected from hydrogen, alkyl, aryl, or R_{b} and R_{c} are taken together with the nitrogen to which they are attached forming a saturated cyclic or unsaturated cyclic system containing 3 to 8 carbon atoms with at least one substituent.

The definition of “heteroaryl” includes, but is not limited to, such specific groups as thienyl, benzothienyl, isobenzothienyl, 2,3-dihydrobenzothienyl, furyl, pyranyl, benzofuranyl, isobenzofuranyl, 2,3-dihydrobenzofuranyl, pyrrolyl, pyrrolyl-2,5-dione, 3-pyrrolinyl, indolyl, isoindolyl, 3H-indolyl, indolinyl, indolizinyl, indazolyl, phthalimidyl (or isoindoly-1,3-dione), imidazolyl, 2H-imidazolinyl, benzimidazolyl, pyridyl, pyrazinyl, pyridazinyl, pyrimidinyl, triazinyl, quinolyl, isoquinolyl, 4H-quinolizinyl, cinnolinyl, phthalazinyl, quinazolinyl, quinoxalinyl, 1,8-naphthyridinyl, pteridinyl, carbazolyl, acridinyl, phenazinyl, phenothiazinyl, phenoxazinyl, chromanyl, benzodioxolyl, piperonyl, purinyl, pyrazolyl, triazolyl, tetrazolyl, thiazolyl, isothiazolyl, benzthiazolyl, oxazolyl, isoxazolyl, benzoxazolyl, oxadiazolyl, thiadiazolyl, pyrrolidinyl-2,5-dione, imidazolidinyl-2,4-dione, 2-thioxo-imidazolidinyl-4-one, imidazolidinyl-2,4-dithione, thiazolidinyl-2,4-dione, 4-thioxo-thiazolidinyl-2-one, piperazinyl-2,5-dione, tetrahydro-pyridazinyl-3,6-dione, 1,2-dihydro-[1,2,4,5]tetrazinyl-3,6-dione, [1,2,4,5]tetrazinanyl-3,6-dione, dihydro-pyrimidinyl-2,4-dione, pyrimidinyl-2,4,6-trione and the like.

The terms “halogen”, “halide” or “halo” refer to fluorine, chlorine, bromine, and iodine.

The term metallo-enzyme refers to any enzyme which activity depends from the presence of a metal ion.

The term “effective amount” of a compound refers to a sufficient amount of the compound that provides a desired effect. This amount may vary from subject to subject, depending on the species, age, and physical condition of the subject, the severity of the type of cancer that is being treated, the particular chemotherapeutic agent used in combination, its mode of administration, and the like. Therefore, it is difficult to generalize an exact “effective amount,” yet, a suitable effective amount may be determined by one of ordinary skill in the art.

The term “pharmaceutically acceptable” refers to a compound, additive or composition that is not biologically or otherwise undesirable. For example, the additive or composition may be administered to a subject along with a compound of the disclosure without causing any undesirable biological effects or interacting in an undesirable manner with any of the other components of the pharmaceutical composition in which it is contained.

The term “pharmaceutically acceptable salts” includes hydrochloric salt, hydrobromic salt, hydroiodic salt, hydrofluoric salt, sulfuric salt, citric salt, maleic salt, acetic salt, lactic salt, nicotinic salt, succinic salt, oxalic salt, phosphoric salt, malonic salt, salicylic salt, phenylacetic salt, stearic salt, pyridine salt, ammonium salt, piperazine salt, diethylamine salt, nicotinamide salt, formic salt, urea salt, sodium salt, potassium salt, calcium salt, magnesium salt, zinc salt, lithium salt, cinnamic salt, methylamino salt, methanesulfonic salt, picric salt, tartaric salt, triethylamino salt, dimethylamino salt, tris(hydroxymethyl)aminomethane salt and the like. Additional pharmaceutically acceptable salts are known to those of skill in the art.

As used herein, the term “patient” refers to organisms to be treated by the methods of the present disclosure. Such organisms include, but are not limited to, humans. In the context of the disclosure, the term “subject” generally refers to an individual who will receive or who has received treatment for the treatment of a disease, disorder or pathology.

According to embodiments of the present disclosure, there are provided compounds having the general structure A or pharmaceutically acceptable salts thereof:

R—X (A)

Examples of some specific compounds that are within the purview of the present disclosure and are described by the general structure A include compounds listed under paragraph [0004] of this application.

Various synthetic schemes can be designed for manufacturing the products having the structure A, including the specific compounds listed above. Synthetic processes can be designed by those having ordinary skill in the art.

Pharmaceutically acceptable salts of the compounds of the present disclosure may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The above-described compounds A, including the sub-genera I can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, isotonic agents may be included, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the methods of preparation include vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds A, including the specie I and its derivatives can be determined by comparing their in vitro activity, and in vivo activity in animal models. Methods for the extrapolation of effective dosages in mice, and other animals, to humans are known to those having ordinary skill in the art who can, for example, be guided by the procedures described in U.S. Pat. No. 4,938,949.

Generally, the concentration of the compounds A, including the species I and derivatives in a liquid composition, such as a lotion, can be between about 0.1 and 25 mass %, such as between about 0.5 and 10 mass %. The concentration in a semi-solid or solid composition such as a gel or a powder can be between about 0.1 and 25 mass %, such as between about 0.5 and 2.5 mass %.

The amount of the compounds A, including the species I and derivatives, or an active salt or derivative thereof, required for use in treatment will vary not only with the particular salt selected but also with the route of administration, the nature of the condition being treated and the age and condition of the patient and will be ultimately at the discretion of the attendant physician or clinician.

### EXAMPLES

The following examples are intended to further illustrate but not limit the scope of the disclosure.

### Example 1

### Synthetic Scheme for the Preparation of Some Compounds of General Formula A

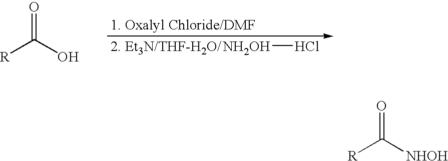

### Example 2

### Synthetic Scheme for the Preparation of Some Compounds of General Formula A

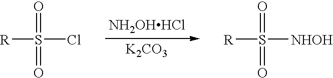

### Example 3

### Synthetic Scheme for the Preparation of Some Compounds of General Formula A

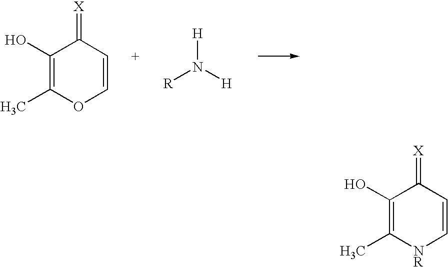

Although the disclosure has been described with reference to the above examples, it will be understood that modifications and variations are encompassed within the spirit and scope of the disclosure. Accordingly, the disclosure is limited only by the following claims.

What is claimed is:

1. A compound having the general structure A or a pharmaceutically acceptable salt thereof:
R—X (A)
wherein R is an alkyl or aryl moiety comprising heterocyclic structures; and
X is a metal chelating group among one of the following:

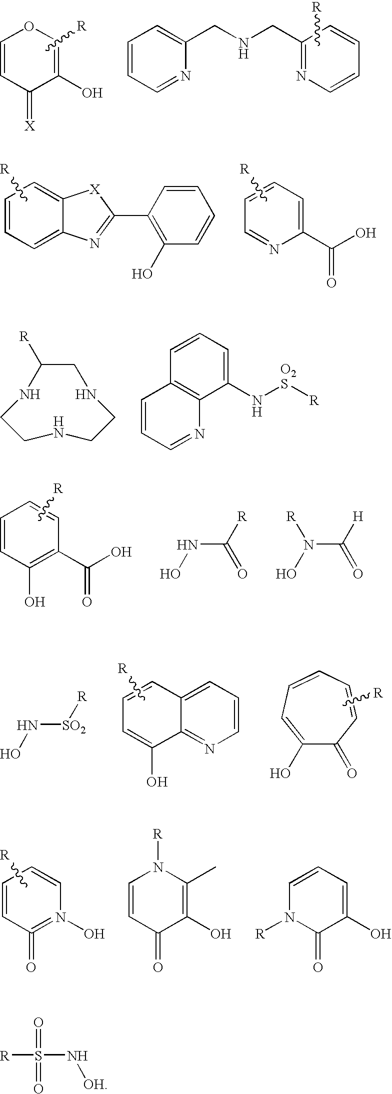

2. A compound of claim 1, wherein the compound binds to a metallo-enzyme thus inhibiting its activity.

3. A method of use of compounds A and derivatives from the general structure of claim 1 as potent and selective metallo-enzyme inhibitors:

4. A pharmaceutical composition comprising a compound of any one of claims 1-3 and a pharmaceutically acceptable carrier therefor.

5. A method of treatment of human malignancies comprising administering a pharmacologically effective dose of a pharmaceutical composition of claim 4 to a subject in need thereof, thereby treating and/or delaying the progression of the disease.

6. A method of making a compound of claim 1, as shown in Examples 1, 2 or 3.
